(19) United States
(12) Patent Application Publication
(10) Pub. No.: US 2019/0285625 A1
(43) Pub. Date: Sep. 19, 2019

(54) BIOSENSOR AND MEASURING METHOD THEREOF

(71) Applicant: ELECTRONICS AND TELECOMMUNICATIONS RESEARCH INSTITUTE, Daejeon (KR)

(72) Inventors: Bong Kyu KIM, Daejeon (KR); Chang-Geun AHN, Daejeon (KR); Kwang Hyo CHUNG, Daejeon (KR); Eun-Ju JEONG, Daejeon (KR); Chul HUH, Daejeon (KR); Seunghwan KIM, Incheon (KR); Jin Tae KIM, Daejeon (KR); Won Kyoung LEE, Daejeon (KR)

(21) Appl. No.: 16/299,438

(22) Filed: Mar. 12, 2019

(30) Foreign Application Priority Data
Mar. 13, 2018 (KR) 10-2018-0029427

(51) Int. Cl. G01N 33/543; B01L 3/00; G01N 33/541

(52) U.S. Cl. CPC G01N 33/54386; B01L 2300/0636; G01N 33/541; B01L 3/5027

(57) ABSTRACT

A biosensor includes a solution chamber containing primary antibodies, secondary antibodies, and measuring substances, an injection fluid transfer unit for connection the solution chamber to an injection port, and a discharging transfer unit for connecting the solution chamber to a discharging port.

### CROSS-REFERENCE TO RELATED APPLICATION

This application claims priority to and the benefit of Korean Patent Application No. 10-2018-0029427 filed in the Korean Intellectual Property Office on Mar. 13, 2018, the entire content of which is incorporated herein by reference.

### BACKGROUND OF THE INVENTION

### 1. Field of the Invention

The present invention relates to a biosensor and a measuring method thereof.

### 2. Description of Related Art

A biosensor is a sensor capable of selectively sensing bio-materials to be analyzed, and generally includes bioreceptors for sensing bio-materials in the sensor. As the biomaterial, an enzyme, a protein, a receptor, a cell, a tissue, and DNA capable of selectively reacting and binding with a characteristic biomaterial are used.

Application fields of biosensors include environmental fields used for measurement of wastewater phenols, heavy metals, pesticides, phosphates, and nitrogen compounds, as well as medical fields such as sensors for early diagnosis and monitoring of blood sugar, diabetes, cancer, and the like. Biosensors are applied to analysis of residual pesticides in food, antibiotics, and infectious germs, and their applications range from military, industrial, and research sensors.

On the other hand, the signal conversion method used to detect biomaterials can be divided into an electrochemical method and an optical method. In the optical method for detecting a biomaterial, a labeled optical biosensor in which an antibody is labeled with a measuring substance and the antigen corresponding to the antibody is detected, and then the amount of the antigen to be analyzed is quantitatively detected in proportion to the intensity of the signal measured from the biosensor, is widely used.

In order to measure small amount of biomarkers with a small antibody size, primary antibodies, secondary antibodies, and materials for measurement are used in the tagged optical biosensor. In general, a labeled optical biosensor first binds the primary antibodies to the antigens and then inserts the secondary antibodies and the measuring substances. Thus, there is a problem that the process such as a plurality of washing processes is complicated.

The above information disclosed in this Background section is only for enhancement of understanding of the background of the invention and therefore it may contain information that does not form the prior art that is already known in this country to a person of ordinary skill in the art.

### SUMMARY OF THE INVENTION

The present invention has been made in an effort to provide a biosensor having a simple process by simplifying the process and reducing the number of times of washing by not adding secondary antibodies, and a measuring method thereof.

According to an exemplary embodiment of the present invention, a biosensor is provided.

The biosensor includes a solution chamber containing immobilized primary antibodies, secondary antibodies bound to measuring substances, an injection fluid transfer unit for connecting the solution chamber to an injection port, and a discharging fluid transfer unit for connecting the solution chamber to a discharging port.

The primary antibodies may be immobilized in the solution chamber.

The secondary antibodies and the measuring substances may be bound to each other and may be present in a solution in the solution chamber.

Each antigen supplied through the injection fluid transfer unit may be simultaneously bound to the primary antibodies and the secondary antibodies.

A concentration of the antigen may be calculated by measuring an amount of binding agents to which the primary antibodies, the secondary antibodies, the antigens, and the measuring substances are bound.

Substances except for the binding agents to which the primary antibodies, the secondary antibodies, the antigens, and the measuring substances are bound may be discharged through the discharging fluid transfer unit by adding washing solution.

Amplifying agents supplied through the injection fluid transfer unit may be bound to the measuring substances.

The measuring substances may be a metal or a chemical material having light transmittance, fluorescent characteristics, or other optical property.

The biosensor may further include a light source and a photodetector.

In another exemplary embodiment of the present invention, a measuring method of a biosensor is provided.

The measuring method of the biosensor includes preparing a solution chamber immobilized primary antibodies, secondary antibodies bound to measuring substances, injecting antigens into the solution chamber, and measuring a concentration of the measuring substances.

The measuring method may further include injecting amplifying agents after the antigen binds to the primary antibodies and the secondary antibodies at the same time.

The primary antibodies may be immobilized in the solution chamber in the measuring method.

The secondary antibodies and the measuring substances may be bound to each other and be present in a solution of the solution chamber in the measuring method.

The measuring substances may be a metal or a chemical material having light transmittance, fluorescent characteristics, or other optical property.

The measuring method may further include, after each antigen binds to the immobilized primary antibodies and the secondary antibodies at the same time, discharging, through washing, substances except for the binding agents to which the primary antibodies, the secondary antibodies, the antigens, and the measuring substances are bound.

### BRIEF DESCRIPTION OF THE DRAWINGS

FIG. 1 shows a biosensor according to an embodiment of the present invention.

FIG. 2 is a flowchart showing a measuring method of a biosensor according to an embodiment of the present invention.

FIG. 3 is a view for explaining a measurement process of the biosensor according to Comparative Example 1.

FIG. 4 is a view for explaining a measurement process of the biosensor according to Embodiment 1.

FIG. 5 is a graph showing storage stability of a biosensor according to an embodiment of the present invention.

### DETAILED DESCRIPTION OF THE EMBODIMENTS

In the following detailed description, only certain exemplary embodiments of the present invention have been shown and described, simply by way of illustration. As those skilled in the art would realize, the described embodiments may be modified in various different ways, all without departing from the spirit or scope of the present invention. Accordingly, the drawings and description are to be regarded as illustrative in nature and not restrictive. Like reference numerals designate like elements throughout the specification.

Throughout this specification and the claims that follow, when it is described that an element is “coupled” to another element, the element may be “directly coupled” to the other element or “electrically coupled” to the other element through a third element. In addition, unless explicitly described to the contrary, the word “comprise” and variations such as “comprises” or “comprising” will be understood to imply the inclusion of stated elements but not the exclusion of any other elements.

A biosensor according to an embodiment of the present invention includes a solution chamber including immobilized primary antibodies, secondary antibodies bound to a substance for measurement, an injection fluid transfer unit for connecting the solution chamber to an injection port, and a discharging fluid transfer unit for connection the solution chamber to a discharging port.

Hereinafter, a biosensor according to an embodiment of the present invention will be described in detail.

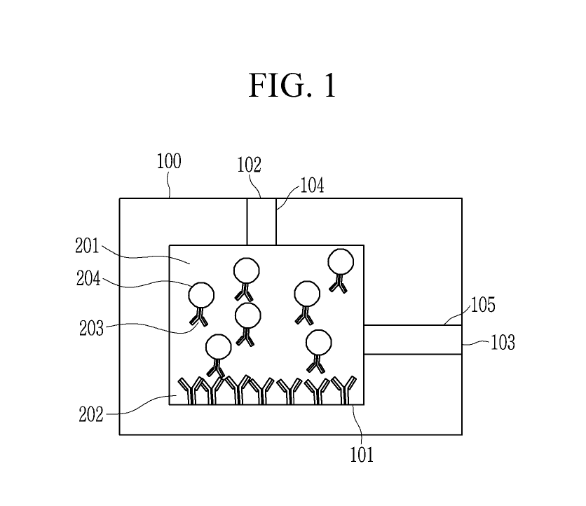

FIG. 1 shows a biosensor 100 according to an embodiment of the present invention.

As shown in FIG. 1, the biosensor 100 according to an exemplary embodiment of the present invention includes a solution chamber 101, an injection fluid transfer unit 104, and a discharging fluid transfer unit 105.

The solution chamber 101 includes immobilized primary antibodies 202, secondary antibodies 203 bound to a measuring substance 204 and further includes a storage solution 201.

The primary antibody 202 and the secondary antibody 203 are different from each other, and each binds to a specific antigen that is a biomarker. The primary antibody 202 was immobilized on the bottom or other side surface of the solution chamber 101, and the measuring substance 204 may be present in the solution chamber 101 in a state of being combined with the secondary antibody 203.

The measuring substance 204 may be an indirect marker for analyzing the concentration of the antigen, for example, an optical marker or a fluorescent marker. For example, when the measuring substance 204 is an optical marker, the concentration of the measuring substance can be quantitatively analyzed by irradiating the biosensor 100 with a light source and measuring the light transmittance of the measuring substance. The measuring substance 204 may be a metal having light transmittance, and examples thereof include, but are not limited to, gold and silver.

The measuring substance 204 may be present in the solution chamber 101 in a state of being combined with the secondary antibody 203. When the antigen binds to the secondary antibody 203, it forms a binding agent with the measuring substance 204 and uncombined measuring substance is washed out through a discharging fluid transfer unit 105, so that the concentration of the antigen can be measured by measuring the amount of the measuring substances 204.

The biosensor 100 according to the embodiment of the present invention includes the immobilized primary antibodies 202 and the secondary antibodies 203 in one solution chamber 101 so that the antigen can specifically bind to the primary antibody 202 and the secondary antibody 203 at the same time when the antigens are injected from the outside. Since the secondary antibody 203 is present in combination with the measuring substance 204 as described above, the concentration of the antigen is calculated by measuring the amount of the binding agents to which the immobilized primary antibody 202, the secondary antibody 203, the antigen (not shown), and the measuring substance 204 are bound. That is, the concentration of the antigen is proportional to the amount of the measuring substances 204 bound to an antigen, a secondary antibody and an immobilized primary antibody.

The antigen, for example, in the form of a plasma sample separated from blood may be injected into an injection port 102, and then injected into the solution chamber 101 through the injection fluid transfer unit 104. Only the specific antigen to be measured among the antigens present in the transferred plasma binds to the primary antibody 202, the secondary antibody 203, and the measuring substance 204.

Since the primary antibody 202 is immobilized on the bottom of the solution chamber 101, when the antigen serving as a biomarker forms a binding agent with the primary antibody 202, the secondary antibody 203, and the measuring substance 204, the binding agent can be likewise immobilized on the bottom of the solution chamber 101.

Therefore, when an antigen serving as a biomarker forms the binding agent with the primary antibody 202, the secondary antibody 203, and the measuring substance 204 and then a washing solution such as water is injected through the injection port 102, the binding agent in which the primary antibody, the antigen, the secondary antibody, and the measuring substance are bound remains and other residual substances are removed through the discharging fluid transfer unit 105 and a discharging port 103.

An amplifying agent may be used to increase the sensitivity of the measurement. The amplifying agent supplied through the injecting fluid transfer unit may specifically bind to the measuring substance. When the amplifying agent used for signal amplification is injected into the solution chamber 101 through the injection port 102, the amplifying agent is bound to the binding agent to which the primary antibody, the antigen, the secondary antibody, and the measuring substance are bound, and then the unbound amplifying agent is removed to the discharging port 103.

Although not shown in FIG. 1, the biosensor 100 according to the embodiment of the present invention may further include a light source and a photodetector. After the binding agent which binds with the primary antibody, the antigen, the secondary antibody, and the measuring substance is formed, if an amplifying agent for signal amplification is injected, the unbound amplifying agent is washed, and then the permeability is measured using, for example, a light source and a photodetector, the amount of the antigens present in the solution chamber 101 can be known and then the concentration of the antigens present in the blood plasma can be accurately measured. However, various measuring methods such as a fluorescence method can be used as the measuring method in addition to the light transmittance change method.

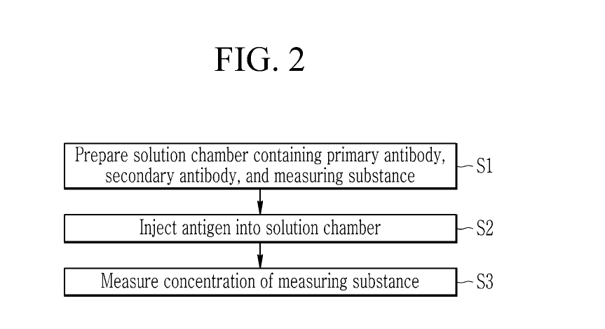

FIG. 2 is a flowchart showing a measuring method of the biosensor 100 according to an embodiment of the present invention.

First, a solution chamber containing primary antibodies immobilized on the bottom, secondary antibodies bound to a measuring substance, storage solution is prepared (S1). —The contents of the primary antibody, the secondary antibody, and the measuring substance are as described above.

Next, the antigens are injected into the solution chamber (S2).

The antigens can be injected into the solution chamber in a state of being contained in plasma excluding blood cells, for example.

The primary antibody and the secondary antibody are different antibodies from each other, and each binds specifically to an antigen that becomes a biomarker. Since the primary antibody is immobilized on the bottom of the solution chamber and the secondary antibody and the measuring substance have binding properties, when the antigen is injected, a binding agent of the primary antibody-antigen-secondary antibody-measuring substance is formed.

In the measuring method of a biosensor according to an embodiment of the present invention, since the antigen, which is a biomarker existing in the injected plasma, is bound to the primary antibody and the secondary antibody at the same time, and the concentration of the antigen can be measured by measuring the amount of the bound primary antibody-antigen-secondary antibody-measuring substance, the antigen, and the secondary antibody, the measuring substances which are unbound are removed by washing.

Then, the concentration of the measuring substance is measured (S3).

The measurement material may be, for example, an optical marker or a fluorescent marker. For example, when the measurement marker is an optical marker, the concentration of the measuring substance can be quantitatively analyzed by irradiating the biosensor 100 with a light source and measuring the light transmittance of the measuring substance. For example, the measuring substance may be a metal having optical transparency, and examples thereof include, but are not limited to, gold and silver.

Although not shown in FIG. 2, the method may further include injecting an amplifying agent after the antigen binds to the primary antibody and the secondary antibody at the same time.

When the amplifying agent used for signal amplification is injected into the solution chamber, the amplifying agent binds to the binding agent of the primary antibody-antigen-secondary antibody-measuring substance. Subsequently, when the washing agent is injected into the injection port, the unbound amplifying agent is removed to the discharging port.

In the above-described measuring method of a biosensor, a secondary antibody is put into a solution chamber and then an antigen is injected. As compared with the conventional method in which a secondary antibody is injected after injecting the antigen and washing, it is possible to reduce the number of processes and also to simplify the measurement process since it is possible to omit the washing step which is carried out after the primary antibody is injected and before the secondary antibody is injected.

Generally, a biosensor requires a long time for the reaction between the antigen and the antibody due to the low concentration of the antigen. However, in the biosensor according to the embodiment of the present invention, the primary antibody and the secondary antibody bind simultaneously, thereby the measurement time can be reduced.

Hereinafter, embodiments of the present invention will be described in detail with reference to examples. The following examples are for illustrative purposes only and are not intended to limit the scope of the invention.

### Measurement Process of Biosensor

### Comparative Example 1

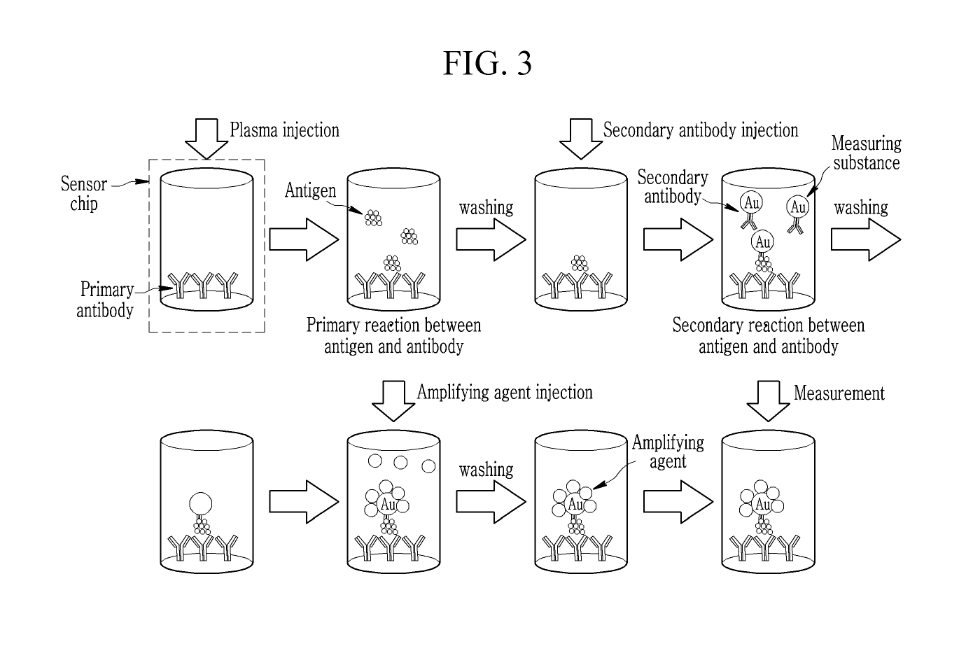

FIG. 3 is a view for explaining the measurement process of the biosensor according to Comparative Example 1.

Referring to FIG. 3, blood plasma from which blood cells are removed is injected into the sensor chip to which the primary antibody is immobilized. The antigen, which is a biomarker existing in the injected plasma, is bound to the primary antibody immobilized on the sensor chip, and unnecessary substances that are not bound and interfere with the measurement are removed through washing. Then, when the binding agent of the secondary antibody and the measuring substance (e.g. Au particles) is injected, it binds to the antigen bound to the primary antibody. When the amount of the bound primary antibody-antigen-secondary antibody-measuring substance is measured, the concentration of the biomarker can be measured, where unbound substances are removed by washing as they interfere with the measurement. In addition, an amplifying agent necessary for amplification is injected to increase the measurement sensitivity. In the case of the amplification agent, the reaction should be proportional to the antigen. Therefore, the amplification agent that is not bound to the antigen must be removed through the washing.

According to the measuring method of the biosensor according to Comparative Example 1, since the process for adding the secondary antibody must be performed separately and the washing step must be performed after the antigen is injected and before the secondary antibody is injected, not only is the process complicated, but also the measurement time is long and the measurement reproducibility is low.

### Embodiment 1

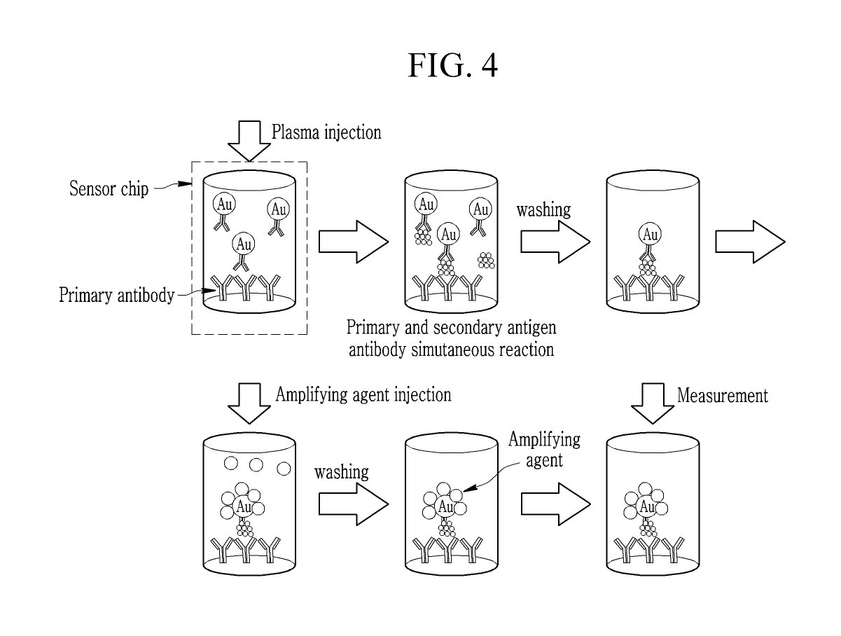

FIG. 4 is a view for explaining a measurement process of the biosensor according to Embodiment 1.

Referring to FIG. 4, unlike Comparative Example 1, Embodiment 1 is intended to simplify the measurement process of the biosensor system through a method in which a secondary antibody and a measuring substance are pre-injected into a sensor chip.

First, the sensor chip in which the primary antibodies are immobilized on the bottom and the secondary antibodies bound to the measuring substance (e.g. Au particles) is prepared. Then, the plasma containing the antigens is injected into the sensor chip to start the measurement of the biosensor system. The antigen, which is a biomarker present in the injected plasma, binds to the primary antibody and the secondary antibody at the same time. Since the concentration of the antigen is measured when the amount of the bound primary antibody-antigen-secondary antibody-measuring substance is measured, the antigen, the secondary antibody, and the measuring substance which are unbound are removed by washing. The subsequent measurement process proceeds in the same manner as the biosensor process of Comparative Example 1.

According to the measuring method of the biosensor according to Embodiment 1, the step such as the insertion of the secondary antibody can be reduced, and the number of times of washing can be reduced, thereby simplifying the measurement process. A general biosensor takes a long time for the reaction between the antigen and the antibody due to the low concentration of the antigen, while the binding between the primary antibody and the secondary antibody in the measuring method of the biosensor according to Embodiment 1 proceeds simultaneously, thereby the measurement time is expected to be reduced by almost half.

### Evaluation of Storage Stability of Biosensor Chip

Unlike the measuring method of the biosensor according to Comparative Example 1 in which the measuring substance bound to the secondary antibody is injected when the biosensor is driven, the measuring method of the biosensor according to Embodiment 1 is characterized in that the primary antibody and the measuring substance bound to the secondary antibody are stored at the same time, so that it was evaluated whether the stability could be deteriorated by the change in the properties of the secondary antibody and the measuring substance.

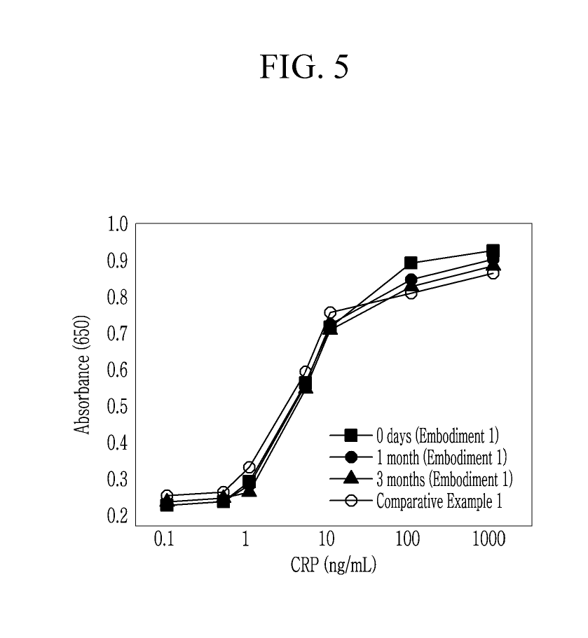

FIG. 5 is a graph showing absorbance according to C-reactive protein (CRP) concentration of the biosensors according to Embodiment 1 and Comparative Example 1 according to a storage period.

Referring to FIG. 5, even in the case of Embodiment 1 in which the primary antibody and the measuring substance bound to the secondary antibody are simultaneously stored in the solution chamber of the sensor chip, it can be seen that it has an absorbency to that in Comparative Example 1 in which the measuring substance bound to the secondary antibody is injected when the biosensor is driven (a result of measurement immediately after sample injection is shown in Comparative Example 1). Therefore, it can be confirmed that the measuring method of the biosensor according to Embodiment 1 has no limitation.

According to an exemplary embodiment of the present invention, it is not necessary to perform the step of separately inserting the secondary antibodies, and the number of times of washing is reduced, so that the concentration of the biomarker can be easily measured.

According to the embodiment of the present invention, it is possible to provide a biosensor capable of simply measuring the concentration of biomarkers by allowing the antigen to simultaneously bind with the immobilized primary antibody and the secondary antibody bound to a measuring substance in the chamber.

While this invention has been described in connection with what is presently considered to be practical exemplary embodiments, it is to be understood that the invention is not limited to the disclosed embodiments. On the contrary, it is intended to cover various modifications and equivalent arrangements included within the spirit and scope of the appended claims.

What is claimed is:

1. A biosensor comprising:
a solution chamber containing primary antibodies, secondary antibodies, measuring substances, and storage solution;
an injection fluid transfer unit for connecting the solution chamber to an injection port; and
a discharging fluid transfer unit for connecting the solution chamber to a discharging port.

2. The biosensor of claim 1, wherein the primary antibodies are immobilized in the solution chamber.

3. The biosensor of claim 1, wherein the secondary antibodies and the measuring substances are bound to each other and are present in a solution in the solution chamber.

4. The biosensor of claim 3, wherein antigens supplied through the injection fluid transfer unit are simultaneously bound to the primary antibodies and the secondary antibodies.

5. The biosensor of claim 4, wherein a concentration of the antigens is calculated by measuring an amount of binding agents to which the primary antibodies, the secondary antibodies, the antigens, and the measuring substances are bound.

6. The biosensor of claim 5, wherein substances except for the binding agents to which the primary antibodies, the secondary antibodies, the antigens, and the measuring substances are bound are discharged through the discharging fluid transfer unit.

7. The biosensor of claim 5, wherein amplifying agents supplied through the injection fluid transfer unit are bound to the measuring substances.

8. The biosensor of claim 1, wherein the measuring substances are a metal or a chemical material having light transmittance, fluorescent characteristics, or other optical property.

9. The biosensor of claim 1, further comprising a light source and a photodetector.

10. A measuring method of a biosensor, comprising:
preparing a solution chamber containing primary antibodies, secondary antibodies, and measuring substances;
injecting antigens into the solution chamber; and
measuring a concentration of the measuring substances.

11. The measuring method of claim 10, further comprising:
injecting amplifying agents after the antigens bind to the primary antibodies and the secondary antibodies at the same time.

12. The measuring method of claim 10, wherein the primary antibodies are immobilized in the solution chamber.

13. The measuring method of claim 10, wherein the secondary antibodies and the measuring substances are bound to each other and are present in a solution of the solution chamber.

14. The measuring method of claim 10, wherein the measuring substances are a metal or chemical material having light transmittance, fluorescent characteristics, or other optical property.

15. The measuring method of claim 10, further comprising:
after the antigens bind to the primary antibodies and the secondary antibodies at the same time, discharging, through washing, substances except for binding agents to which the primary antibodies, the secondary antibodies, the antigen, and the measuring substances are bound.
